(19) United States
(12) Patent Application Publication
(10) Pub. No.: US 2007/0286916 A1
(43) Pub. Date: Dec. 13, 2007

(54) Synbiotic Use

(75) Inventor: Stig Bengmark, Hoganas (SE)

Correspondence Address: MERCHANT & GOULD PC, P.O. BOX 2903, MINNEAPOLIS, MN, 55402-0903, US

(73) Assignee: Synbiotics AB, Kagerods (SE)

(21) Appl. No.: 10/588,574

(22) PCT Filed: Feb. 17, 2005
(86) PCT No.: PCT/SE05/00220

§ 371 Date: May 31, 2007

(30) Foreign Application Priority Data
Feb. 17, 2004 (SE) 0400355-4

(51) Int. Cl. A61K 35/74; A61P 29/00; C12P 39/00

(52) U.S. Cl. 424/780; 435/42

(57) ABSTRACT

The invention relates to the use of at least two lactic acid bacterial strains selected from the group comprising Pediococcus pentosaceus 16:1 (LMG P-20608), Leuconostoc mesenteroides 23-77:1 (LMG P-20607), Lactobacillus paracasei subsp paracasei F-19 (LMG P-17806), and Lactobacillus plantarum 2362 (LMG P-20606) for the manufacturing of a formulation for the prevention or treatment of stress-induced inflammatory disorder.

### FIELD OF INVENTION

The invention relates to the use of at least two lactic acid bacterial strains selected from the group comprising Pediococcus pentosaceus 16:1 (LMG P-20608), Leuconostoc mesenteroides 23-77:1 (LMG P-20607), Lactobacillus paracasei subsp paracasei F-19 (LMG P-17806), and Lactobacillus plantarum 2362 (LMG P-20606) for the manufacturing of a formulation for the prevention or treatment of stress-induced inflammatory disorder.

### BACKGROUND OF THE INVENTION

Animals and especially human beings are in the modern Society increasingly exposed to stress of various kinds. Such stress will most often result in an increased expression of certain immune system-related components in the body, which induces severe inflammation in various tissues of the body. Such an inflammation will significantly reduce the capacity of the innate (natural) immune system and thereby decrease the individual resistance to disease, which makes the mammal prone to develop subsequent infections, but also, if prolonged stress, chronic diseases of various kinds. Some of these diseases are difficult to treat, and impossible to heal, as the existing medical medicaments for treatment of such diseases is much limited. This is especially true for chronic diseases, which constitute an increased burden to the Society. But also the treatment of acute conditions, such as infections is limited. Antibiotics for example have increasingly lost their ability to prevent and cure infections and septic manifestations such as systemic inflammatory syndrome (SIRS) and multiple organ failure (MOF), and might in addition lead to progress of antibiotic resistant infections. As far as can be judged, no new and more effective antibiotics are in the pipeline to reach the market. All presently existing applications to US Food and Drug Administration (FDA) are only for smaller modifications of existing antibiotic preparations. No applications are for new antimicrobial agents. Clearly the pharmaceutical companies are not presently able to produce new and challenging concepts. Less and less antimicrobials are approved. FDA approved nine in 1998-2003 and mainly within existing classes (cephalosporins and fluoroquinolones), compared to 16 between 1983-1987. The picture is not very different for prevention and treatment of chronic diseases. Although more new pharmaceuticals are introduced for prevention and treatment of chronic diseases their potential to cure are generally much limited, and most importantly they are often associated with toxicity and development of severe side effects.

The truth is that the morbidity and mortality in chronic as well as acute diseases, especially in the so called severely sick patients or critically ill patients have, despite claimed progress in medico-pharmaceutical and surgical treatments remained unaffected or increased during the last few decades. Much support that modern pharmaceuticals this far has been largely unable to stem the tide of these conditions. Especially the incidence of chronic diseases is fast increasing in the developed and but increasingly also in the developing world. A recent statistics from the World Health Organization (WHO) suggests that chronic diseases constitute 46% of global disease burden and 59% of global deaths. Each year approximately 35 million people will die in chronic diseases. The epidemy of chronic diseases is closely associated with Western life style; physical and mental stress, reduced physical activity and consumption of refined, calorie-condensed fatty, sugary and starchy foods in combination with reduced intake of fibre- and antioxidant-rich fresh fruits and vegetables, but also bacteria. The secondary morbidity and mortality in association with advanced surgical and medical treatments, and in medical and surgical emergencies (physical injuries and acute diseases), already unacceptably high is also increasing at a worrying rate, and affects mainly those with an incipient or manifest chronic disease. Sepsis is the most common complication both to advanced medical and surgical treatments. It is estimated that annually in the US, 751 000 are affected by severe sepsis, leading to death in 215 000 patients (29%), and making sepsis the tenth most common cause of death (see Hoyert D L, Arias E, Smith B L et al. National Vital Statistics Reports (serial on line) www.cdc.gov/nchs/data/nvsr/nvsr49/nvsr49 08pdf, and Angus DC, Linde-Zwirble W T, Lidicker J et al. (2001) Epidemiology of severe sepsis in the United States: Analysis of incidence, outcome and associated costs of care. Crit Care Med 29:1303-1310).

Central to the development of chronic diseases, but also influencing acute diseases, is the occurrence of a condition called metabolic syndrome (characterised by obesity, hypertension, insulin resistance, glucose intolerance and fatty infiltration of almost all organs, particularly the liver. Metabolic syndrome is a pre-stage of chronic diseases and a condition which today affects about 25% of the population in countries like the US and the UK, and another 25 to 50% are borderline/at risk to develop the condition. There is evidence that this condition is associated with over-consumption of calorie-condensed refined agricultural products and under-consumption of less calorie-condensed fruits, vegetables and live bacteria. Our Paleolithic forefathers consumed up to ten times as much plant fibres, fifty times more antioxidants, and billion times more of beneficial non-pathogenic microorganism than modern man.

Various producers of various supplements with live bacteria/probiotics do often claim health benefits from supplying such formulations. Most of the probiotics on the market, do not survive the acidity of the stomach, or the bile acid content of the small intestine, nor do they adhere to colonic mucosa and even temporary colonise the stomach. And no health benefits can be demonstrated. This was recently demonstrated when a standard commercial product, TREVIS™ (Chr Hansen Biosystem, Denmark), containing lb acidophilus LA5, bifidobacterium lactis BP12, streptococcus thermophilus, lb bulgaricus and combined with 7.5 g oligofructose was supplied to patients in two separate controlled studies, one involving critically ill patients (Jain P K, McNaught C E, Anderson A D G, et al. Influence of synbiotic containing Lactobacillus acidophilus LA5, Bifidobacterium lactis BP12, Streptococcus thermophilus, Lactobacillus bulgaricus and oligofructose on gut barrier function and sepsis in critically ill patients: a randomized controlled trial. Clinical Nutrition 2004; 23:467-475), and the other postoperative patients (Woodcock N P, McNaught C E, Morgan D R, et al. An investigation into the effect of a probiotic on gut immune function in surgical patients. Clin Nutr. 2004;23:1069-1073). Although treatment in both studies favourably influenced the microbial composition of the upper gastrointestinal tract, did it not influence intestinal permeability, nor was it associated by measurable clinical benefits).

There is a need of finding compositions, which can be used to prevent the development of both acute and chronic diseases in mammals, such as human beings and animal. Thereby the costs as well as the suffering will be reduced or eliminated. Additionally, less mammals will develop chronic diseases. The present formulation has shown a significant ability to improve the immune system and strengthen the body's resistance to diseases.

### SUMMARY OF THE INVENTION

The invention relates to the use of at least two lactic acid bacterial strains selected from the group comprising Pediococcus pentosaceus 16:1 (LMG P-20608), Leuconostoc mesenteroides 23-77:1 (LMG P-20607), Lactobacillus paracasei subsp paracasei F-19 (LMG P-17806), and Lactobacillus plantarum 2362 (LMG P-20606) for the manufacturing of a formulation for the prevention and/or treatment of stress-induced inflammatory disorder. The formulation may be a pharmaceutical formulation.

By the use of that particular formulation it is possible to revert a stress-induced inflammatory disorder in a mammal and thereby reduce/eliminate the possibility that the stress-induced inflammatory disorder will continue into a chronic disease stage, and thereby become permanent. The formulation has the ability to revert most of the stress-induced alterations in body function, such as those being defined under definitions.

The invention also relates to, a method of treating a mammal suffering from a stress-induced inflammatory disorder.

The invention provides a completely new way of preventing and/or treating a mammal suffering from a stress-induced inflammatory disorder which in the case where the disorder is not treated it mostly continue into a chronic disease. Thereby the mammal will be cured in a fast, efficient and low expensive way and the need of future medication for a chronic disease evolved from the stress-induced inflammatory disorder eliminated or reduced. Thereby the health costs of an individual will be reduced for the society.

### DETAILED DESCRIPTION OF THE INVENTION

Definitions

In the context of the present application and invention the following definition apply:

The term “stress-induced inflammatory disorder” is intended to mean a phenomenon, in which a mammal, upon stress, increase the secretion of pro- and anti-inflammatory cytolines and acute phase proteins, increased infiltration of affected tissues with neutrophils, increased myeloperoxidase activity in the tissues affected, and increased oxidation of vital tissues, and increased accumulation in the tissues of oxidation products such as malonedealdehyde. Such an induced inflammation if not treated as soon as possible will continue into a chronic disease, and contribute to death.

The term acute phase reaction” (APR) is intended to mean a series of complex reactions which occur in the body under stress, mental or physical; infection, trauma, surgical operation, advanced medical treatment or delivery—all reactions aimed to provide optimal protection against progress of disease. They involve the whole body, but especially the central nervous system, the hypothalamus and the hypophysis, which via the so called neuro-endocrine axis activates all the organs in the body, particularly the adrenals, the thyroids, the gonads, the liver, the gut, its mucosa and lymphatic system but also to a large extent the intestines, the intestinal mucosa and the intestinal flora. It is estimated that there is more nerve endings in the gut, which affects the intestine and flora in stress conditions, compared to other places within the body. This intimate connection between the central nervous system (CNS) and the gut makes the gut deeply involved in the progress of disease from its earliest moments.

The increased secretion of norepinephrine in stress is shown to especially activate potentially pathogenic microorganisms (PPMs) in the gut, which as a consequence dramatically increase their disease-inducing ability/virulence. These normally indolent colonisers of the intestine will under stress change their phenotype and become life-threatening pathogens. Central to the APR is an enormously elevated release of various pro- and anti-inflammatory cells, to a large extent from cells which are not regarded as immune cells such as mucosal, endothelial and fat cells/adipocytes, but also by the flora, itself. Important is also the release of fibrinolysis-regulating substances, such as plasminogen activator inhibitor type 1 (PAI-1), which impairs the viscosity of blood, increase the coagulation, reduces the nutritive blood flow to the critical organs, such as the brain, the lungs and the liver, and often leads to formation of clots/thromboses. Characteristic to APR is increase in temperature, chill, sonmolence, anorexia and profound changes in blood levels of plasma proteins, lipids, minerals, hormones, cytokines as well as cellular elements.

The term “chronic phase reaction” (CPR) is intended to mean a phenomenon in which the mammal suffers from chronic fatigue, asthenia, reduced appetite, reduced physical activity, reduced mood, sometimes mental depression and reduced muscle mass, while the changes in chemical and cellular parameters, although obvious, are more discrete.

Common to APR and CPR is hypermetabolism, increased hepatic glycogenesis (production of sugar by the liver), increased glucose turnover in the body, reduced muscle uptake of glucose, hyperlipidemia (too much fat in the circulation) and increased lipolysis of adipose tissues (breakdown of fat tissues and mobilisation to the circulation of), especially visceral fats, increased production of non-esterified fatty acids (NEFAs), increased protein synthesis in the liver and increased protein turnover in the body, increased blood glucose levels, increased insulin secretion and insulin resistance. Especially fibrinogen and PAI-1 are significantly elevated in CPR, and fibrinolysis (solubilisation/disintegration of formed clots) significantly impaired.

The formulation of the invention has the ability to modulate/regulate: most often and in most organs inhibit, but occasionally also stimulate a number of different molecules involved in inflammation including phospholipase, lipooxygenase, cyclooxygenase 2 (COX-2), leulcotreines, thromboxane, prostaglandins, nitric oxide, collagenase, elastase, hyaluronidase, monocyte vchemoattractant protein-1 (MCP-1), interferon-inducible protein, tumour necrosis factor (TNF), and interleukins such as interleukin 1 (IL-1), interleukin 6 (IL-6), interleukin 10 (IL-10) and interleukin 12 (IL-12), but also other interleukins. Central to the function of the formulation/medicament is inhibition of nuclear factor κB (NF-κB), inducible nitric oxide synthase (iNOS), and inhibition of COX-2 gene expression. An important function is also often an increased delivery to and availability in the tissues of pyruvate and of oxygen.

Description

It is well-known that when a mammal is under stress certain processes are triggered aimed to control the stress. Examples of functions, which are influenced by stress are among many others cytokines, myeloperoxidase activity, malondealdehyde accumulation and increase in accumulation of neutrophils in the affected tissues. When the stress continue over a longer period the mammal, will develop an inflammatory state, and become sensitive to develop other disease manifestations, chronic diseases and severe, often life-threatening infections.

Our invention relates to the finding that by the use of at least two lactic acid bacterial (LAB) strains selected from the group comprising Pediococcus pentosaceus 16:1 (LMG P-20608), Leuconostoc mesenteroides 23-77:1 (LMG P-20607), Lactobacillus paracasei subsp paracasei F-19 (LMG P-17806), and Lactobacillus plantarum 2362 (LMG P-20606) it is possible to reduce and/or eliminate the influences of all the negative changes of acute phase reaction and chronic phase reaction, as defined above, including neutrophil infiltration, overproduction of cytokines, increased myeloperoxidase activity in tissues, and accumulation of pro-oxidants and oxidation products in the tissues. Hereby it is possible to prevent and/or treat stress-induced inflammatory disorders.

According to one embodiment the invention relates to a composition consist of the four above, mentioned LAB, or more.

The lactic acid bacteria (LAB) to be used are selected after extensive studies of >350 human fecal bacteria and about 180 bacteria harvested from fresh growing rye.

After extensive studies of numerous functions of each lactic acid bacterium were the four most bioactive LAB (with ability to modulate inflammation) chosen for the composition. Special consideration was given to unique and superior abilities to survive in the low pH of the stomach and in the high bile acid content of the small intestine, unique ability to attach to colonic mucosa and to temporary colonise the large intestine, high capacity to ferment various types of plant fibres, including rather fermentation resistant fibres, such as inulin, a balanced production of both pro- and anti-inflammatory molecules, such as cytokines, strong ability to produce several bioactive molecules, especially heat shock proteins and their production of significant amounts of antioxidants.

The specific abilities of the composition, and the ability of the strains to potentiate each others bioactivity makes it possible to inhibit and revert stress-induced inflammatory disorders at an early stage of development and thereby reduce the possibility that the mammal, later will develop severe chronic disease.

The bacterial strains used have been deposited pursuant to, and in satisfaction of, the requirements of the Budapest Treaty on the International Recognition of the Deposit of Microorganisms for the Purposes of Patent Procedure with the Belgian Coordinated Collection of Microorganisms (BCCM), Gent, Belgium, under Accession No. LMG P-20608 for Pediococcus penosaceus 16:1, Accession No. LMG P-20607 for Leuconostoc mesentorides 23-77:1, Accession No. LMG P-17806 for Lactobacillus paracasei (paracasei) F-19, and Accession No. LMG P-20606 for Lactobacillus plantarum 2362, also sometimes referred to as Lactobacillus plantarum 2592.

The bacterial strains may be administrated together with a pharmaceutically acceptable liquid component, but also as a powder or built into plasters or wound protecting tissues. The bacterial strains may be present in a concentration at least 10⁶ CFU/ml, such as at least 10⁷, 10⁸, 10⁹, 10¹⁰ or at least 10¹¹ CFU/ml.

According to certain embodiments one or more acceptable liquid component may be needed. Such components are well known to those skilled in the art. Preferably, distilled water or buffered aqueous media are used, which contain pharmaceutically acceptable salts and buffers. Suitable salt solutions are PBS, PBSS, GBSS, EBSS, HBSS, and SBF. The liquid component can also be of a more hydrophobic nature in dependence of the application.

The formulation may further comprise one or more therapeutic agents, such as an agent against the stress-induced inflammatory disorder.

Accordingly the formulation may comprises the above mentioned four bacterial strains or even more, as long as the above mentioned bacterial strains are present. The formulation of the invention being used for to prevent or treat a mammal suffering from a stress-induced inflammatory disorder, such as a human being or an animal.

The inflammatory disorders prevented or treated by the formulation may be chest/lung inflammation, urinary inflammation, vaginal inflammation, bowel inflammation, lever inflammation, stomach inflammation, muscle inflammation and brain inflammation. However, the inflammation may also be of another origin.

Accordingly the formulation may further comprise at least one fibre. The combination of utilising fibre, such as plant fibres may release from the fibres numerous nutrients, antioxidants, growth-, coagulation and other factors, which in the body will modulate innate and eventually also adaptive immune defence mechanisms, hereby improving the body's ability to resist disease development. Fibres from fruits and vegetables have strong bioactivities by themselves. This is to maintain mucosal growth and functions, to maintain water and electrolyte balance, to provide energy and nutrients for the host, to provide energy and nutrients for the flora and to provide resistance against invading pathogens, which is further enhanced by the addition of the specific LAB as above. The fermentation process in the lower gastrointestinal tract releases through action of microbial enzymes numerous nutrients and antioxidants, but also various growths and coagulation-controlling as well as inflammation-controlling molecules. Antioxidants, but also other nutrients released in the lower GI tract by flora if existing and the supplied specific LAB, have significant pro-regenerative, antibacterial, antithrombotic, vasodilatory, anti-inflammatory and anti-carcinogenic effects. Nitric oxide is also released by fermentation by the LAB described above and is important for several bodily functions such as gastro-intestinal motility, blood flow, ventilation (dilatory effects) but also control of pathogenic microorganisms.

Examples of fibres are beta-glucan, inulin, pectin, resistant starch, cellulose, hemicellulose, arabinoxylans, arabinogalactans, polyfructose, inulin, oligofructans, galacto-oligosacharides, gums, mucilages, pectins, dextrins, maltodextrins, potato dextrins, synthesised carbohydrates, polydextrose, methylcellulose, hydroxypropylmethylcellulose. Other examples from plants are fibres is selected from lignin substances from plants selected from the group comprising waxes, cutin, phytate, saponin, suberin and tannins.

Specific examples of polymeric plant carbohydrates are beta-glucan, inulin, pectin and resistant starch. By combining the four above mentioned lactic acid bacteria and the four fibres in the composition a unique and strong bioactivity as well a synergistic functions in the body are obtained. When supplied in combination, the components of this synbiotic composition show strong synergistic/potentiated health benefits both in experimental animals and in humans. A significant increase in these functions was observed when the chosen fibres were included in the composition.

Accordingly the formulation of the invention comprises at least one antioxidant, vitamin, mineral, amino acid, peptide or protein. Examples of such substances are antioxidant and vitamins, minerals, such as selenium and zinc, amino acids such as glutamine and arginine, and various peptides. When the content in the added fibres of specific antioxidants or nutrients are insufficient pure substances can be added, including synthetic versions thereof. Numerous plant fibres, nutrients, antioxidants and vitamins may be considered: such as vitamin C (from various fruits and vegetables, vitamin E (from among others various grains, nuts and vegetarian oils, flavonols such as quercitin (from among others onion, apple, grapes, berries and broccoli), epigallocatechin (from among others tea leaves, especially green tee), epicatechin (from among others black grapes and red wine) flavonoids such as anthocyanidins (from among others various grapes, raspberries, strawberry, aubergine), oenine (from among others black grapes and red wine), hydrocinnamates such as p-coumaric acid from among others white grapes and tomatoes, spinach, asparagus and cabbage), ferulic acid (from among others grains, particularly oat, tomatoes, spinach, asparagus, and cabbage) carotenoids such as lycopene(such as from tomatoes), β-Carotene (among others from carrots, sweet potato, tomatoes, and paprika (pepper), such as from yellow paprika- pepper, xanthophylls such as β-cryoxanthine (among others rich in mango, papaya, peaches, paprika/pepper and oranges) glutathione (among others in broccoli (flower), Parsley (leaf), spinach, yellow squash, potato, tomato green paprika/pepper, tangerine, cauliflower and orange), folic acid (among others rich in yeast, beans, lentils, wheat germs, wheat bran, spinach, peanuts), resveratrol (red grape, red wine and peanuts and curcumin (turmeric), anti-oxidative minerals such as selenium and zinc.

All these active antioxidants and minerals are released in the intestine, particularly the large intestine, by fermentation of the flora. Severely stressed persons have lost their entire flora. Supply of the composition restores the flora, and access to the important food-born antioxidants and other nutrients. Extra strong effects have been obtained by combining antioxidants such as glutathione, folic acid, resveratrol and particularly curcumin with the composition.

Additionally, the formulation may comprise glutamine or a synthetic version thereof.

The invented formulation may be in the solid or liquid form such as tablet, gel spray, capsules or granulates. The form of the formulation is dependent on the use. The administration route of the formulation may be any route such as orally, ingested such as by tube fed, intraperitoneal, intramuscular or subcutaneous injection, or in case of tumors, intra-tumorally.

Use of the Formulation

The invented formulation can be used to treat stress-induced inflammatory disorders, either acute or chronic. The stress may be induced by for example physical trauma, physical harm, an accident, burns, childbirth or poisoning or other forms of non-specified stress-induced trauma.

Examples of disorders, which can be prevented, include chest infections, such as those caused by the stress of reduced availability of oxygen (hypoxia), too high exposure to oxygen (hyperoxia), too high levels of sugar in blood (hyperglycemia), too low level of sugar in blood (hypoglycemia), irradiation, exposure to toxic chemical including certain pharmaceuticals and especially chemotherapeutic agents, trauma, pancreatitis, cystic fibrosis, viral, microbial, fungal and other infections and other pulmonary diseases and childbirth.

The formulation may be used in stress-induced inflammatory disorders in the bowel and is also effective against inflammation induced by bacteria such as Helicobacter, Clostridium difficile, HIV, rotavirus but also other microbial infections.

Additionally, the formulation may be used for preventing and treating postoperative morbidity. Such postoperative disorders are for example those caused by postoperative infections, which are triggered by stress-induced inflammation, most often in the lungs, but also in other organ systems, such as the urinary tract. In this connection immunomodulatory effects have been documented from supplementation of the composition. The ability of these lactic acid bacteria to produce bioactive substances such as immunomodulins and beta-defensins are important properties of the formulation. These substances have the ability to down-regulate the exaggerated pro-inflammatory effect of pro-inflammatory cytokines, such as TNF and IL-6, but also other cytokines and pro-inflammatory substances such as acute phase proteins and oxidation-promoting substances such as homocysteine.

Examples of groups of acute diseases, spontaneous or induced by advanced medical or surgical treatments, where the use of the formulation is beneficial are bone marrow or other transplantations, extensive medical and surgical treatments, thermic injuries, acute infections and general septic disorders.

Thus, the use of the formulation according to the invention constitutes an inexpensive and powerful tool, which has no side effects, and the formulation can be used for long-term treatment of patients with a chronic disease.

Finally the formulation may be used for the treatment of a mammals, suffering from a stress-induced inflammatory disorder, such as a human being or animal, such as domestic animals.

### EXAMPLES

The following illustrative examples show the unique documented effects obtained by using the medicament according to the invention.

### Example 1

### Formulation

One formulation was prepared consisting of Pediococcus pentosaceus 16:1 (LMG P-20608), Leuconostoc mesenteroides 23-77:1 (LMG P-20607), Lactobacillus paracasei subsp paracasei F-19 (LMG P-17806), and Lactobacillus plantarum 2362 (LMG P-20606) at a concentration of 10¹⁰ CFU/ml of each bacteria and 2.5 g of each beta-glucan, inulin, pectin and resistant starch (Ljung et al., 2002, Microb, Ecol, Health Dis, 3 (suppl):4 and Kruszewska et al., 2002, Microecol Ther 29:37.

The formulation was produced by Medipharm AB, K\dot{a}geröd, Sweden using the protocol by Ljung et al., as mentioned above.

### Example 2

### Effect in Intensive Therapy (ITU) Patients

A study was performed in ten ITU patients. Samples were taken for bacterial cultivation from all 10 patients. After anaerobic incubation for 48 hours at 37° C. presence of LAB was attempted using API 50CHL identification strips (bioMerieux). In seven of the ten patients no growth of LAB could be detected.

Supplementing with the composition of four lactic acid bacteria strains (Pediococcus pentosaceus 16:1 (LMG P-20608), Leuconostoc mesenteroides 23-77:1 (LMG P-20607), Lactobacillus paracasei subsp paracasei F-19 (LMG P-17806), and Lactobacillus plantarum 2362 (LMG P-20606) and four polymeric carbohydrates (beta-glucan, inulin, pectin and resistant starch) for three days returned the LAB level in feces to the level about 10⁷. 18 patients in ITU received the composition and were compared to 18 patients receiving placebo. The infection rate and mortality was in of half in the treated patients.

### Example 2

### Effect in Severe Trauma Patients

100 multiple injured patients were prospectively randomised to intragastric feeding with: glutamine (group A), soluble fibres (group B), peptide (group C) and a synbiotic composition of lactic acid bacteria (LAB) and fermentable fibers (as defined in example 1) (group D). IP was evaluated by measuring lactulose-mannitol (L/M) excretion on days 2, 4 and 7. Intestinal permeability (IP), by repeated measurement of lactulose-mannitol (L/M) excretion, and clinical outcome were studied. The IP increased continuously up to day 7 in all groups, except the group supplemented the above synbiotic formulation (group D), in which the L/M index from 0.439 (0.224-0.626) on day 4 significantly (p<0.05) dropped to 0.128 (0.088-0.320) on day 7. L/M index increased significantly (p<0.02) in group A from 0.061 (0.010-0.379) on day 2 to 0.223 (0.076-0.705) on day 4 and was 0.515 (0.332-1.153) on day 7. Thirty four of totally 48 infections observed were chest infections, its incidence being significantly (p<0, 03) lower in the group group treated with the synbiotic formulation (group D) than in the other groups. The patients supplemented with the symbiotic formulation did better than the others; had lower intestinal permeability, less translocation of pathogenic microorganisms and less chest infections (pneumonia).

### Example 3

### Effect on Development of Adhesions and Thrombosis

A study was performed in thirty-six Wistar albino rats were divided into three groups, all subjected to a standard peritoneal adhesion model. During 3 weeks before the production of adhesions were the animals supplied by gavage with the composition of four lactic acid bacteria strains and four polymeric carbohydrates as defined in example 1 and a placebo consisting of the four polymeric carbohydrates, respectively. Seven days following the induction of adhesions the numbers and extent of adhesions were evaluated as well as two parameters of fibrinolysis: tissue-type plasminogen activator (tPA) and plasminogen activator inhibitor type-1 (PAI-1) measured on biopsies from undamaged parietal peritoneum.

The adhesion score in the treated group was 1.6±0.60 compared to 3.55±13.07 in the control (P=0.0001) The mean tPA level in the treatment groups was 3.65±0.88 compared to 5.12±11.88 in the control group (P=0.007) and the PAI-1 in the treated group 24.93±7.67 compared to 18.23±4.41 in the control group (3 P=0.004).

When in an additional similar study extensive antibiotic treatment was tried, no similar benefits were obtained.

### Example 4

### Effect in Abdominal Cancer Operations

A prospective randomised double-blind trial was undertaken in 30 patients undergoing abdominal cancer operations. All patients received enteral nutrition with supply of a composition of four lactic acid bacteria strains and four polymeric carbohydrates. A comparison was made between one group receiving the composition and another group receiving a standard clinical nutrition.

The incidence of postoperative bacterial infections during the first month after operation was 6.7% compared to 47% in the control. Laboratory studies performed on days 3 and 6 after the operation demonstrated significant improvement parameters such as prealbumin, C-reactive protein, serum cholesterol, white cell blood count and serum endotoxin. The first defecation occurred significantly earlier in the group that had been treated with the composition according to the invention.

### Example 5

### Effect in Transplant Patients

A prospective randomised double-blind trial was undertaken in 66 liver transplant recipients. A comparison was made between one group receiving the synbiotic composition of four lactic acid bacteria strains and four polymeric carbohydrates and a group receiving only the four bioactive polymeric carbohydrates therein. The treatment started the day before surgery and continued for 14 days.

The incidence of postoperative bacterial infections was significantly reduced from 48% with only fibres to only 3% (one of 33 patients developed a slight urinary infection). Bacterial cultures showed microbial growth in 8 of the treated patients and 16 of the control patients. The most frequently identified microorganisms were gut-derived, with a predominance of Enterococcus faecalis and faecium observed.

In addition, three patients undergoing bone marrow transplantation have been treated with the composition according to the invention. No episodes of infection were observed. The in-hospital time was record short. In addition it was observed that the lymphocyte counts normalised quicker than normally seen.

### Example 6

### Effect on Inflammation and Infection in Chronic Liver Disease

Western people, especially those, who are obese and suffer metabolic syndrome, have also fatty livers. Fatty livers are especially seen in persons with visceral obesity. Visceral fat cells (adipocytes) have an increased expression of cytokines, especially of TNFα. The amount of fat in the abdomen can vary from a few milliliters to about six liters, which can explain the increased exposure of pro-inflammatory molecules, such as TNFα, in adipose individuals. Increased release of pro-inflammatory molecules, such as TNFα, together with an over-expression of γ-interferon and underexpression of IL-10 sensitises the liver to both endotoxins and to toxic effects, especially of TNFα, which most likely is a key factor behind the progressive liver damage seen in patients with liver cirrhosis.

The supply of the four lactic acid bacterial strains (probiotics, as defined in example 1) alone or in combination with the four polymeric carbohydrates (synbiotics, as defined in example 1) have the ability not only to reduce the production and absorption of endotoxin in the intestine, but also to down-regulate the production of pro-inflammatory cytokines, including TNFα. Furthermore, the composition induces in patients with liver disease significant improvements in bilirubin and prothrombin activity as well as in albumin level.

A long-term supply of a composition of four lactic acid bacteria strains and four polymeric carbohydrates reduces both the inflammation of the liver, the fatty infiltration of the liver (steatosis) and retards the progress of liver destruction.

When the synbiotic composition of four lactic acid bacteria strains and four polymeric carbohydrates was supplied to eleven patients with chronic liver disease a significant reduction in production of TNFα by peripheral blood mononuclear cells in response to stimulation by endotoxin or Staphylococcus aureus enterotoxin B (SEB), was reduced to half in comparison to pre-supplementation levels in the majority of cirrhotic patients, who were supplied with the composition.

### Example 7

### Effect on Gut Colonisation, Liver Function and Degree of Encephalopathy in Chronic Liver Disease

The synbiotic composition of four lactic acid bacteria strains and four polymeric carbohydrates was supplied during one month to patients with chronic liver disease and results compared to 15 similar patients, who received a placebo (non-fermentable, non-absorbable fibre) during the same time period.

The intestinal pH was significantly reduced in the treatment group compared to placebo. Significant decreases in the number of Escherichia coli, Staphylococcus and Fusobacterium were observed. Significant decreases in ammonia(s), levels of endotoxin(s) and ALT (expression of impaired liver function) were observed in the group receiving the synbiotic composition, but not in the placebo group. The improvements in liver function observed in the group supplemented the synbiotic composition (as defined in example 1) were accompanied by significant improvements also in psychometric tests and in degree of encephalopathy.

### Example 8

### Effect on Liver Blood Flow and Indocyanine Clearance

Indocyanine clearance (ICG_{R15}) test, a measurement of liver blood flow, was performed in 15 patients.

A supplementation with the synbiotic composition of four lactic acid bacteria strains (as defined in example 1) and four polymeric carbohydrates (as defined in example 1) resulted in a significant reduction in ICG_{R15} in the cirrhotic patients, an improvement most likely due to a reduced swelling of endothelial and sinusoidal cells and hereby reduced resistance to flow.

Accordingly, a long-term supplementation of the synbiotic composition has the potential to reduce the number and the degree of severity of bleeding episodes in patients with liver cirrhosis.

### Example 9

### Effect in Colitis Patients

Ten patients with moderate colitis were treated during two weeks with twice daily doses of the synbiotic composition of four lactic acid bacteria strains (as defined in example 1) and four polymeric carbohydrates (as defined in example 1) reconstituted in 120 ml normal saline. Studies were repeated on days 0, 7,14 and 21. Significant reduction in diarrhoea scores was observed at days 7, 14, and 21. Visible blood in stool was significantly reduced at days 14 and 21. Also nocturnal diarrhoea, urgency to defecation and consistency of stool were significantly reduced at days 7, 14, and 21.

A stronger effect obtained with the four specific lactic acid bacteria strains, especially when combined with the four specific polymeric carbohydrates.

### Example 10

### Effect on Helicobacter Colonisation of the Stomach

Ten animals and six humans with Helicobacter infection in the stomach received, the composition of four lactic acid bacteria strains and four polymeric carbohydrates (as defined in example 1) for 14 days. A total elimination of Helicobacter colonies was observed.

### Example 11

### Effects on Chest Infections

Several patients with cystic fibrosis have been treated with the composition of four lactic acid bacteria strains and four polymeric carbohydrates. There have all been in a rather late stage of disease, loosing weight, suffering diarrhea, and on almost constant antibiotic treatment. They have all made a dramatic turn around, diarrhea has been controlled, gained weight, and need of antibiotics been eliminated. The state of disease has improved. The synbiotic composition of four lactic acid bacteria strains (as defined in example 1) and four polymeric carbohydrates (as defined in example 1) has also been used in combination with the antioxidant curcumin (turmeric) and total disappearance of disease manifestations observed.

### Example 12

### Effects when Administered Subcutaneously

Abdominal infection was induced in experimental animals by a procedure called cecal ligation and puncture (CLP). This initiates normally a general sepsis in the body most often if untreated with fatal outcome. The lungs are early affected by inflammation, which is a serious complication. This inflammation is manifested in accumulation of white blood cells (neutrophils) and increase in inflammatory enzymes such as myeloperoxidase and oxidation substances such as malonedialdehyde. The tables below demonstrate how treatment with the formulation inhibits these manifestations. Similar changes are also observed in other organs such as skeleton muscles and the intestinal walls. Also these are reduced, by supplementing the formulation.

group-1: CLP—supplemented formulation during 3 days before CLP

group-2: CLP—supplemented formulation during 1 day before CLP

group-3: CLP—supplemented saline during 3 days before CLP

group-4: CLP—supplemented saline during 1 day before CLP

group-5: Only formulation for 3 days

group-6: Only formulation for 1 day

group-7: CLP—no other treatment

group-8: control—sham operation, no other treatment

Reduced infiltration of neutrophils, activity of myeloperoxidase, and content of malonedealdehyde is demonstrated in the groups supplemented the formulation, groups 1 and 2, compared to the groups supplemented saline (groups 3 and 4)

Neutrophil Counts of Lung Tissues

| Group 1 | Group 2 | Group 3 | Group 4 | Group 5 | Group 6 | Group 7 | Group 8 |
|---|---|---|---|---|---|---|---|
| 3 | 27 | 53 | 52 | 8 | 9 | 53 | 8 |
| 5 | 29 | 52 | 48 | 7 | 9 | 47 | 11 |
| 5 | 35 | 49 | 47 | 7 | 8 | 52 | 9 |
| 4 | 31 | 53 | 55 | 6 | 9 | 51 | 12 |
| 5 | 36 | 53 | 56 | 8 | 6 | 46 | 10 |
| 4 | 29 | 54 | 54 | 9 | 9 | 59 | 10 |
| 5 | 34 | 51 | 50 | 10 | 8 | 53 | 11 |
| 5 | 33 | 50 | 49 | 9 | 7 | 52 | 14 |
| 3 | 30 | 48 | 48 | 8 | 9 | 50 | 12 |
| 4 | 28 | 48 | 49 | 9 | 7 | 48 | 9 |

Myeloperoxidase Activity of Lung Tissues

| Group 1 | Group 2 | Group 3 | Group 4 | Group 5 | Group 6 | Group 7 | Group 8 |
|---|---|---|---|---|---|---|---|
| 7915.00 | 8446.00 | 9782.00 | 9701.00 | 3321.00 | 3245.00 | 8834.00 | 3623.00 |
| 6340.00 | 6975.00 | 17988.00 | 14096.00 | 2202.00 | 2100.00 | 19008.00 | 1802.00 |
| 2269.00 | 4733.00 | 13623.00 | 15800.00 | 2878.00 | 1590.00 | 11123.00 | 1544.00 |
| 3587.00 | 9790.00 | 12756.00 | 13208.00 | 2756.00 | 2370.00 | 12566.00 | 2769.00 |
| 4541.00 | 5599.00 | 9850.00 | 10988.00 | 1995.00 | 1708.00 | 9997.00 | 1877.00 |
| 3066.00 | 5148.00 | 5337.00 | 4899.00 | 2979.00 | 3540.00 | 6211.00 | 3666.00 |
| 2766.00 | 6381.00 | 5223.00 | 5893.00 | 2110.00 | 2045.00 | 5112.00 | 2049.00 |
| 4742.00 | 9965.00 | 16786.00 | 16800.00 | 2522.00 | 2002.00 | 17123.00 | 2032.00 |
| 5964.00 | 2263.00 | 5436.00 | 4900.00 | 2267.00 | 2544.00 | 4987.00 | 2540.00 |
| 4336.00 | 2331.00 | 12300.00 | 11505.00 | 3655.00 | 3673.00 | 13465.00 | 3533.00 |
| Mean ± SD | | | | | | | |
| 4552 ± 1767 | 6163 ± 2724 | 10908 ± 4631 | 10779 ± 4385 | 2668 ± 549 | 2481 ± 752 | 10842 ± 4823 | 2543 ± 813 |

Malondealdelhyde (MDA) in Lung Tissues (Marker of Oxidative Injury)

| Group 1 | Group 2 | Group 3 | Group 4 | Group 5 | Group 6 | Group 7 | Group 8 |
|---|---|---|---|---|---|---|---|
| 0.446263 | 0.484565 | 0.513470 | 1.208315 | 1.062237 | 0.358545 | 0.581921 | 0.524563 |
| 0.411756 | 0.365444 | 0.787732 | 0.725497 | 0.942031 | 0.433309 | 0.671827 | 0.346437 |
| 0.573666 | 0.623434 | 1.311272 | 0.681363 | 0.532158 | 1.064978 | 0.486280 | 0.593927 |
| 0.411756 | 0.553424 | 0.409964 | 0.530185 | 0.171901 | 0.360455 | 1.126020 | 0.593233 |
| 0.784278 | 0.361221 | 0.825072 | 0.823435 | 0.143319 | 0.891682 | 1.530641 | 0.249897 |
| 0.535573 | 0.989229 | 0.601140 | 0.571453 | 0.627312 | 1.959333 | 0.350484 | 1.422626 |
| 0.655465 | 0.412747 | 0.446484 | 0.682802 | 0.674644 | 0.440322 | 0.381991 | 0.358545 |
| 0.694706 | 0.367324 | 0.684679 | 0.549414 | 0.616222 | 0.531148 | 3.443016 | 0.433309 |
| 0.398298 | 0.926354 | 1.062237 | 0.680694 | 0.301253 | 0.207084 | 0.179142 | 1.064978 |
| 0.424373 | 0.666323 | 0.532138 | 0.570168 | 1.462977 | 0.466765 | 0.322011 | 0.360450 |
| MEAN | | | | | | | |
| 0.5336137 | 0.5750065 | 0.7174188 | 0.7023326 | 0.6534036 | 0.6713621 | 0.9073333 | 0.5947965 |

### Example 13

### Effects when Administered Intraperitoneally

The same experiments as in example 12 was performed, but the formulation/medicament applied intraperitonelly. Identical changes were observed.

### Example 14

### Effects when Administered Intramuscularly

The same experiments as in example 12 was performed, but the formulation/medicament applied intramuscularly. Identical changes were observed.

### Example 15

### Effects when Administered by Inhalation

The same experiments as in example 12 was performed, but the formulation/medicament applied by inhalation. Identical changes were observed.

### Example 16

### Effects when Administered by Ingestion/Oral Supply

The same experiments as in example 12 was performed, but the formulation/medicament applied by oral/enteral supply. Identical changes were observed.

### Example 17

### Effects Irradiation/Chemotherapy-Induced Stress

36 patients, with cancers in different organs varying from gastrointestinal to lung and prostatic cancer and undergoing irradiation/chemotherapy suffered in connection with their first treatment cycles severe side effects with fatigue, feeling of sickness, depressed mood, indisposition, vomiting, severe diarrhoea, and dizziness. They were on subsequent treatment cycles treated with the formulation/medicament and all the manifestations either eliminated or radically reduced.

### Example 18

### Effects of the Formulation/Medicament in Patients with Clostridium difficile, HIV and Rotavirus Infections

Eighteen patients, children and adults, who suffered from manifest Clostridium difficile infections with chronic diarrhoea were supplemented daily with the medicament for minimum 30 days. All symptoms were relieved and no signs of Clostridium difficile observed on repeated examination of stool.

Eight patients with manifest HIV, preferably children and suffering weight loss, severe diarrhoea and repeat infections, received for two weeks to 20 months oral daily intake of the medicament. They report increased well-being, reduced incidence of secondary infections, normalisation of stool frequency and consistency, and increased body weights.

Five children suffering rotavirus diarrhoea received, in addition to liberal supply of fluid and salts also an oral supply of the medicament. The symptoms including diarrhoea disappeared within 2 days from institution of treatment.

### Example 19

### Effects when Applied Topically in Burns, Chronic Leg Ulcers and Bed Sores and Around Skin-Penetrating Foreign Materials.

Thirty-five patients with infected burns on skin were treated with a gel of the synbiotic composition of four lactic acid bacteria strains and four polymeric carbohydrates. A dramatic reduction of infection and cleaning of the burned surfaces was observed. An improved healing could also be noted. In five patients the composition was used with further addition of curcumin and additional positive effects observed. Five patients with vaginitis were treated with a gel of the composition of four lactic acid bacteria strains and four polymeric carbohydrates. An instant cure was observed.

Eleven patients with infections around entrances of foreign material through the skin (trachostomies, infusion lines, drainages) were treated topically with a gel of the composition of four lactic acid bacteria strains and four polymeric carbohydrates. A quick elimination of the biofilm and cleaning of the skin around the foreign material was observed. In three patients curcumin was added with further improvement in healing.

### Example 20

### Effects on Ventilation Volume when Inhaled

A significant increase in ventilatory volume was observed in 8 experimental animals on inhaling the four lactic acid bacteria strains dissolved in saline as a composition. Accordingly, the composition can be used for cleaning of mucosal linings in the lungs, and for the dilatation of the air ways.

### Example21

### Effects in Individuals with Rheumatic Disorders

Nine individuals with severe manifestations of rheumatoid arthritis, children and adults, were supplemented orally for 2 months to three years with the formulation/medicament. They report decreased pain, decreased need of pain killers and other drugs, reduced joint swelling, increased flexibility of joints and improved well-being.

### Example 22

### Effects in Chronic Renal Disease

Nine individuals with chronic renal disease and undergoing either hemodialysis or peritoneal dialysis (CAPD) were supplemented daily for 2 months to 3 years with the formulation/medicament. They reported significant improvement in well-being, increase in: appetite, caloric intake, body weight, mid-arm circumference and in serum albumin, less sensibility to infections, and significant reduction in serum cholesterol and serum free fatty acids, and signs improved liver function, and coagulation.

### Example 23

### Effects in Stress-Induced Malfunction of the Bowel such as Irritable Bowel Syndrome (IBS)

Eleven patients with what they regard handicapping bowel syndrome, discomfort, pain, and gas problems received during six weeks to three years a daily oral supply of the medicament. They report reduced pain and general discomfort, normalisation of stool consistency and frequency and reduced problems with gas.

1. A method for manufacturing a formulation for the prevention or treatment of stress-induced inflammatory disorder comprising providing Pediococcus pentosaceus 16:1 (LMG P-20608), Leuconostoc mesenteriodes 23-77:1 (LMG P-20607), Lactobacillus paracasei subsp paracasei F-19 (LMG P-17086), and Lactobacillus plantarum 2362 (LMG P-20606) wherein the bacterial strains are in an amount of at least 10¹¹ CFU/ml of each of the bacteria and at least four different fibres.

2. A method according to claim 1, wherein stress-induced inflammatory disorder is determined as an increase in neutrophils, cytokines, myeloperoxidase and/or accumulation of the oxidation-related malonedealdehyde.

3. A method according to claim 1, wherein the inflammatory disorder is lung inflammation, urinary inflammation, vaginal inflammation, bowel inflammation, stomach inflammation, liver inflammation, muscle inflammation, inflammation of endocrine and reproductive organs, and brain inflammation.

4. A method according to claim 3, wherein the fibre is selected from the group consisting of beta-glucan, inulin, pectin, resistant starch, cellulose, hemicellulose, arabinoxylans, arabinogalactans, polyfructose, inulin, oligofructans, galacto-oligosacharides, gums, mucilages, pectins, dextrins, maltodextrins, potato dextrins, synthesized carbohydrates, polydextrose, methycellulose and hydroxypropylmethlcellulose.

5. A method according to claim 1, wherein the four fibres are inulin, beta-glucan, pectin and resistant starch.

6. A method according to claim 5, wherein the fibres are present in an amount of 2.5 g of each fibre.

7. A method according to claim 4, wherein the fibre is selected from lignin substances from plants selected from the group comprising waxes, cutin, phytate, saponin, suberin and tannins.

8. A method according to claim 1, wherein the formulation further comprises at least one antioxidant, vitamin, mineral, amino acid, peptide or protein.

9. A method according to claim 1, wherein the formulation further comprises glutamine, or a synthetic version thereof.

10. A method according to claim 1, wherein the formulation further comprises one or more therapeutic agents.

11. A method according to claim 1, wherein the formulation is solid or liquid, such as tablet, gel or spray.

12. A method for treating a mammal suffering from a stress-induced inflammatory disorder, comprising applying a formulation to the mammal, the formulation comprising a result of providing Pediococcus pentosaceus 16:1 (LMG P-20608), Leuconostoc mesenteriodes 23-77:1 (LMG P-20607), Lactobacillus paracasei subsp paracasei F-19 (LMG P-17086), and Lactobacillus plantarum 2362 (LMG P-20606) wherein the bacterial strains are in an amount of at least 10¹¹ CFU/ml of each of the bacteria and at least four different fibres.

13. A method according to claim 12, wherein the mammal comprising an animal or a human being.
